(19) United States
(12) Patent Application Publication
(10) Pub. No.: US 2026/0273748 A1
(43) Pub. Date: Sep. 17, 2026

(54) CONTROLLING A JOINT IN A ROBOTIC ARM

(71) Applicant: CMR Surgical Limited, Cambridge (GB)

(72) Inventors: Edward James Wildin Tucker, Cambridge (GB); David Moore, Cambridge (GB); Johnathan Hung, Cambridge (GB)

(73) Assignee: CMR Surgical Limited, Cambridge, OT (GB)

(21) Appl. No.: 18/996,605

(22) PCT Filed: Aug. 14, 2023
(86) PCT No.: PCT/GB2023/052136

§ 371 Date: Jan. 17, 2025

(30) Foreign Application Priority Data
Aug. 15, 2022 (GB) 2211906.9

(51) Int. Cl. B25J 9/16; A61B 34/30; B25J 13/08

(52) U.S. Cl. CPC B25J 9/1674; A61B 34/30; B25J 9/163; B25J 9/1653; B25J 13/085; G05B 2219/40599; G05B 2219/45117

(57) ABSTRACT

A controller for controlling the configuration of a joint in a surgical robot, the joint being driven by a drivetrain which transfers power from a drive source to the joint, the controller being configured to receive a drive source configuration signal indicating a configuration of the drive source and receive a joint configuration signal indicating a configuration of the joint. The controller being configured tot determine a configuration error from a difference in configurations indicated by the drive source configuration signal and the joint configuration signal and determine a configuration error limit from a measured joint torque of the joint and a joint stiffness of the joint. The controller being configured to compare the determined configuration error and the determined configuration error limit and output a fault signal where the determined configuration error equals or exceeds the determined configuration error limit.

### CROSS-REFERENCE TO RELATED APPLICATIONS

This application is the U.S. national stage application of International Application No. PCT/GB2023/052136, filed on Aug. 14, 2023, which international application was published on Feb. 22, 2024, as International Publication WO 2024/038259 A1 in the English language. The international application is incorporated herein by reference, in its entirety. The international application claims priority from GB Patent Application No. 2211906.9, filed on Aug. 15, 2022, which is incorporated herein by reference, in its entirety.

### FIELD OF THE INVENTION

This invention relates to controlling a joint in a robotic arm, for example to controlling the configuration of a joint in a surgical robotic arm.

### BACKGROUND

A typical robotic manipulator comprises a series of rigid elements that are coupled together by one or more joints, which may be joined in series to form an arm. The manipulator further comprises a base unit at a first end of the series of rigid elements, and an end effector at a second end that opposes the first end. Each joint of the manipulator is driven by a drive source and a corresponding drivetrain so as to cause relative motion of the rigid elements. This relative motion is used to vary the configuration of the end effector at a desired location. Each joint may provide rotational motion and/or linear motion. The drive source may be any suitable means, such as an electronic motor or a hydraulic actuator.

In theoretical modelling, the drivetrain that actuates a robotic joint can be modelled as being “stiff”. A stiff drivetrain is one that does not experience any stiction or backlash. Backlash refers to the lost motion that is experienced due to a change in direction and is caused by gaps or clearance between interfacing components within the drivetrain. If there is no stiction or backlash in the drivetrain, then the torque generated by the drive source (i.e. the input torque that is provided to a first end of the drivetrain) will always be proportional to the torque that is experienced by the joint (i.e. output torque at a second end of the drivetrain that opposes the first end).

In reality, the components of the drivetrain that actuate a robotic joint have an associated level of elasticity. This elasticity can cause a mismatch between a driven joint position and an actual joint position effected by the drive source via the drivetrain. Detecting mismatches can cause faults to be registered, which can affect operation of the system.

It is desirable to provide a system that can accommodate drivetrain elasticity to increase operational capability of the system.

### SUMMARY

This summary is provided to introduce a selection of concepts that are further described below in the detailed description. This summary is not intended to identify key features or essential features of the claimed subject matter, nor is it intended to be used to limit the scope of the claimed subject matter.

According to an aspect of the present invention, there is provided a controller for controlling the configuration of a joint in a surgical robot as claimed in claim 1. According to another aspect of the present invention, there is provided a method of controlling the configuration of a joint in a surgical robot as claimed in claim 15. According to another aspect of the present invention, there is provided a controller configured to perform a method as defined herein.

According to another aspect of the present invention, there is provided a control system for controlling the configuration of a joint in a surgical robot as claimed in claim 17. According to another aspect of the present invention, there is provided a method for controlling the configuration of a joint in a surgical robot as claimed in claim 27. According to another aspect of the present invention, there is provided a control system configured to perform a method as defined herein.

According to another aspect of the present invention, there is provided a non-transitory computer readable storage medium having stored thereon computer readable instructions that, when executed at a computer system, cause the computer system to perform a method as defined herein.

Additional aspects of the present invention are defined in the dependent claims.

Any feature of any aspect above can be combined with any one or more other feature of any aspect above. Any method feature may be rewritten as an apparatus feature, and vice versa.

### BRIEF DESCRIPTION OF THE DRAWINGS

The present invention will now be described by way of example with reference to the accompanying drawings.

In the drawings:

FIG. 1 illustrates the configuration of a robot arm;

FIG. 2 is a block diagram illustrating an idealised control loop for controlling the configuration of a joint in a robot arm;

FIG. 3 illustrates the characterisation of the non-linear stiffness of a drivetrain for a robotic joint;

FIG. 4 is a block diagram illustrating a full state control scheme for controlling the configuration of a joint in a robot arm;

FIG. 5 is a block diagram illustrating a control algorithm for a controller as illustrated in FIG. 4, the control algorithm using torque sensor data as an input;

FIG. 6 illustrates the relationship between configuration error limits and torque;

FIG. 7 illustrates another example of the relationship between configuration error limits and torque;

FIG. 8 is a flow chart illustrating a method of controlling the configuration of a joint in a surgical robot;

FIG. 9 is a flow chart illustrating another method of controlling the configuration of a joint in a surgical robot; and

FIG. 10 is a block diagram illustrating components of an exemplary computing-based device.

### DETAILED DESCRIPTION

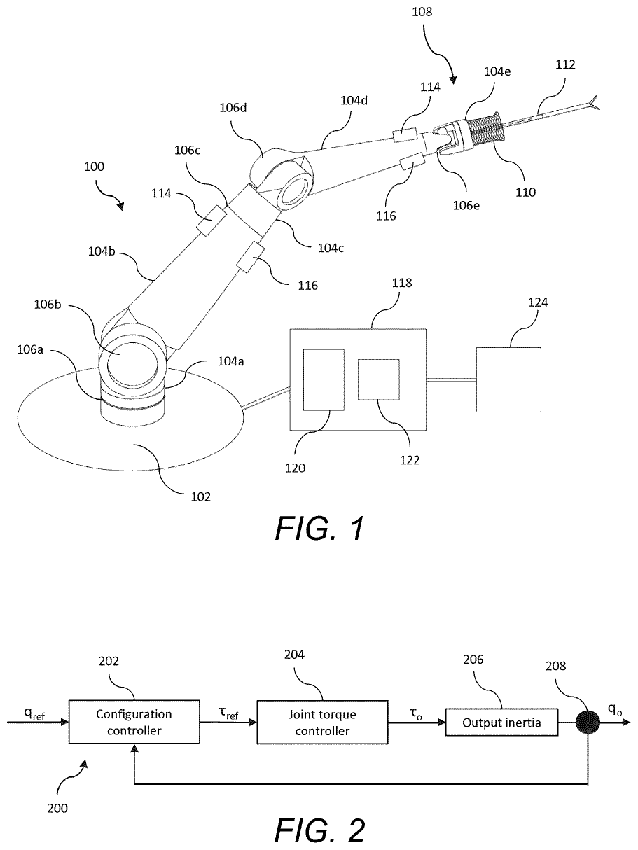

The following description is presented by way of example to enable a person skilled in the art to make and use the invention. The present invention is not limited to the embodiments described herein and various modifications to the disclosed embodiments will be apparent to those skilled in the art. Embodiments are described by way of example only. The following description describes the present techniques in the context of surgical robotic systems, though the features described below are not limited to such systems, but may be applied to robotic systems more generally. In some examples, the present techniques may be applied to robotic systems that operate remotely. Robotic systems can include manufacturing systems, such as vehicle manufacturing systems, parts handling systems, laboratory systems, and manipulators such as for hazardous materials or surgical manipulators. The robotic system illustrated in FIG. 1 is specifically a surgical robotic system. However, the features described below are not limited to such a system but are applicable to robotic systems more generally.

FIG. 1 illustrates a surgical robot having an arm 100 which extends from a base unit 102. The arm comprises a plurality of rigid limbs 104a-e which are coupled by a plurality revolute joints 106a-e. The revolute joints 106a-e are configured to apply rotational motion. Instead of or in addition to these revolute joints, the surgical robot may comprise one or more joints for providing linear motion. The limb that is closest to the base 102 is the most proximal limb 104a and is coupled to the base by a proximal joint 106a. The remaining limbs of the arm are each coupled in series by a joint of the plurality of joints 106b-e. A wrist 108 may comprise four individual revolute joints. The wrist 108 couples one limb (104d) to the most distal limb (104e) of the arm. The most distal limb 104e carries an attachment 110 for a surgical instrument 112. Each joint 106a-e of the arm 100 has one or more drive sources 114 which can be operated to cause rotational (and/or linear) motion at the respective joint. Each drive source 114 is connected to its respective joint 106a-e by a drivetrain which transfers power from the drive source to the joint. In one example the drivetrain comprises one or more gears. In a more specific example, the drivetrain is a harmonic drive. A harmonic drive comprises the concentric arrangement of a wave generator, a flex spline and a circular spline, and increases the torque that can be applied to the joint by the drive source.

In one example, the drive sources 114 comprise motors. The drive sources 114 may comprise hydraulic actuators, or any other suitable drive means. Each joint 106a-e further comprises one or more sensors 116, which can comprise a configuration and/or a force sensor 116. The one or more sensors 116 provide information regarding the current configuration and/or force at that joint. Where the joint is a revolute joint, one or more of the sensors 116 may comprise a torque sensor. Where the joint is configured to provide linear motion, the one or more sensors 116 may comprise a strain gauge. The one or more sensors 116 may comprise a temperature sensor, a current sensor and/or a pressure sensor. Thus, the one or more sensors 116 may provide information regarding the temperature, current and/or pressure (such as hydraulic pressure). In one example, the one or more sensors 116 comprise a position sensor. That is, the sensor 116 may measure the physical position of the joint. In such an example, the one or more sensors 116 may comprise an encoder. An encoder is a position sensor which transforms a position measurement into an electronic signal. The encoder may comprise a linear encoder. The encoder may comprise a rotary encoder.

The drive sources 114 are arranged proximally of the joints whose motion they drive, to improve weight distribution. For clarity, only some of the drive sources and sensors are illustrated in FIG. 1. It can also be appreciated that the surgical robot 100 may comprise more or fewer limbs and joints than those illustrated in FIG. 1.

The surgical robot illustrated in FIG. 1 comprises a single robot arm. Other robot systems such as surgical robot systems may comprise a plurality of robots and/or a plurality of robot arms. For example, other example robot systems such as surgical robot systems may comprise a robot with a plurality of robot arms that can each receive and manipulate a tool such as a surgical instrument, or they may comprise a plurality of robots that each have a robot arm that can receive and manipulate a tool such as a surgical instrument.

The instrument 112 of the surgical robot 100 comprises an end effector or tool for performing an operation. The end effector may take any suitable form. For example, the end effector may comprise smooth jaws, serrated jaws, a gripper, a pair of shears, a needle for suturing, a camera, a laser, a knife, a stapler, a cauteriser or a suctioner. The instrument further comprises an instrument shaft and an articulation located between the instrument shaft and the end effector. The articulation comprises several joints which permit the end effector to move relative to the shaft of the instrument.

Configuration controllers for the drive sources 114 and sensors 116 are distributed within the robot arm 100. The controllers are connected via a communication bus to a control unit 118. The control unit 118 comprises a processor 120 and a memory 122. The memory 122 stores, in a non-transient way, software that is executable by the processor 120 to control the operation of the drive sources 114 to cause the arm 100 to operate. In particular, the software can control the processor 120 to cause (for example via distributed controllers) the drive sources to exert drive in dependence on inputs from the sensors 116 and from a surgeon command interface 124. The control unit 118 is coupled to the drive sources 114 for driving the drive sources in accordance with outputs generated by execution of the software. The control unit 118 is electrically connected to the sensors 116 for receiving sensed input from the sensors, and to the command interface 124 for receiving input from the command interface. The electrical connections described herein may, for example, comprise electrical and/or optical cables, and/or may comprise a wireless connection.

The command interface 124 comprises one or more input devices whereby a user can request motion of the end effector in a desired way. The input devices, or input controllers, could, for example, be manually operable mechanical input devices such as control handles or joysticks, touch-operable inputs such as touchscreens, or contactless input devices such as optical gesture sensors or voice sensors. The input devices might monitor eye movement to receive an input. The input devices could, for example, be some combination of these types of input devices. Commands input by the input devices can include movement commands, for example to move the instrument in a particular way, such as a lateral movement and/or a rotation. Such commands can include end effector commands, for example to control an end effector coupled to a distal end of the instrument 112 to operate the end effector, such as to open/close gripper jaws or to operate (turn on or off) an electrosurgical end effector.

The software stored in the memory 122 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at a surgeon console which suitably comprises the command interface 124 can control the instrument 112 to move in such a way as to perform a desired surgical procedure. Thus, the robot arm 100 functions as a master-slave manipulator where the control unit 118 acts as a master controller. The control unit 118 and/or the command interface 124 may be remote from the arm 100. Where respective control units 118 are provided for each of multiple arms, the control unit 118 can be considered to be a robot arm controller.

The surgical arm 100 can comprise a controller 115 for a joint 106. Suitably, the surgical arm comprises respective controllers 115 for each of the plurality of joints 106. The controller can be termed a joint controller 115. A single such joint controller 115 is illustrated in FIG. 1, but it will be understood that such joint controllers 115 can be provided for two or more joints 106 of the arm 100. The joint controller 115 is coupled to the sensor 116. The joint controller 115 is configured to receive signals from the sensor 116 and to determine a characteristic of the joint from the sensor output, for example the joint configuration or position. The joint controller 115 is coupled to the drive source 114. The joint controller 115 can be configured to receive signals from the drive source, thereby to determine an output of the drive source. The joint controller 115 is suitably configured to determine, from the drive source, a driven configuration or position of the joint 106.

FIG. 2 illustrates an idealised control loop 200 for controlling the configuration of a revolute joint in a robot arm, such as the robot arm illustrated in FIG. 1. The control loop comprises a configuration controller 202, a torque controller 204, an inertia output 206 and a sensor 208.

The configuration controller 202 may be distributed within a robot arm, such as the arm illustrated in FIG. 1, such that it is proximal to a joint that it is configured to control. The sensor 208 can comprise more than one sensor, for example the sensor 208 can comprise at least one sensor for each of two or more joints of the arm. Suitably, the sensor 208 comprises at least one sensor for each joint of the arm. The joint controller 115 may form part of the configuration controller 202. The configuration controller 202 is electrically connected to the sensor 208 by means of a feedback loop. The feedback loop enables the configuration controller 202 to receive measurements indicating the configuration of the joint from the sensor. The configuration controller 202 is also connected to the control unit 118 by means of a feed forward loop and is configured to receive a reference configuration value q_{ref} from the control unit 118. In one example, the configuration controller 202 is a separate entity to the control unit 118. In another example, the configuration controller 202 and the control unit 118 are comprised within the same entity. Thus, in this example, the control unit may be directly connected to the sensor 208.

The reference configuration q_{ref} may otherwise be referred to as the desired or commanded configuration of the joint. The reference configuration q_{ref} is calculated from an end effector pose that is commanded by the surgeon at the command interface 124. The reference configuration q_{ref} may be calculated by applying one or more inverse kinematic calculations to the commanded end effector pose. In one example, the reference configuration q_{ref} is the desired physical position of the joint.

The configuration controller 202 is configured to perform a calculation using the reference position value q_{ref} received from control unit 118 and the feedback value from the sensor 208 to generate a reference torque value τ_{ref}. The reference torque value τ_{ref} indicates the demanded torque that is required to move the joint to the reference configuration q_{ref}. The reference torque value τ_{ref} is then transmitted to the torque controller 204.

The torque controller 204 is electrically connected to the configuration controller 202 and is configured to receive the reference torque value τ_{ref}. From this value of τ_{ref}, the joint torque controller is configured to calculate a value of desired current that is to be generated by the drive source. This desired current is used to generate an output torque τₒ, which is the actual torque that is experienced by the joint when it is driven by the drive source. In an idealised system, the drivetrain that transfers torque from the drive source to the joint is “stiff” and so no torque is “lost” as it is transferred from the drive source to the joint. Thus, the output torque τₒ that is experienced at the joint in such an idealised system is equal to the reference torque τ_{ref} that is received by the configuration controller 202.

A joint that is not stiff will, when it is driven, experience an inertia which opposes the output torque τₒ provided by the drive source. The inertia may be caused by a number of factors that are either internal or external to the joint. An exemplary cause of inertia is the weight of the joint due to gravity. Another exemplary cause is the weight of an object being supported by or interfacing with the joint. Due to the presence of inertia, the configuration of the joint at its output, qₒ, may differ from the reference configuration q_{ref}. For a joint that is stiff, the inertia will not have an effect on the output configuration of the joint qₒ. Thus, for a stiff joint, there will be no difference between the output configuration qₒ and the desired configuration q_{ref}.

The sensor 208 is provided to measure the output configuration qₒ of the joint. The sensor 208 may correspond to one or more of the sensors 116 illustrated in FIG. 1. Thus, the sensor 208 may be comprised within a joint of the robot arm 100. In one example, the output configuration qₒ is the output position of the joint. The sensor 208 may comprise a position sensor such as a linear encoder or a rotary encoder. In this example, the sensor 208 is configured to measure the position of the joint within which it is comprised, and to transmit an electrical signal indicating this measurement to the configuration controller 202. The transmittance of this electrical signal forms a feedback loop that is used by the configuration controller 202 to calculate an updated value of the reference torque τ_{ref}.

Generally, feedback terms are used within control systems to indicate a deviation between the desired configuration of a joint and its actual configuration, and to compensate for disturbances that may take the joint further from steady state. Thus, the value of the reference torque τ_{ref} that incorporates feedback from the sensor 208 may be continuously updated as continuous data measurements are obtained from the sensor 208. The feedback loop may further comprise one or more filters, integrators or differentiators for manipulating the raw output value that is obtained from the sensor 208 before it is provided to the configuration controller 202.

As mentioned above, the joint to be controlled by the loop illustrated in FIG. 2 has been modelled as “stiff”. For stiff joints, the input torque (i.e. the torque that is measured at, or at a position proximal to, a first end of the drive train at which the drive source is located) is proportional to the output toque (i.e. the torque that is measured at, or at a position proximal to, a second end of the drive train at which the joint is located).

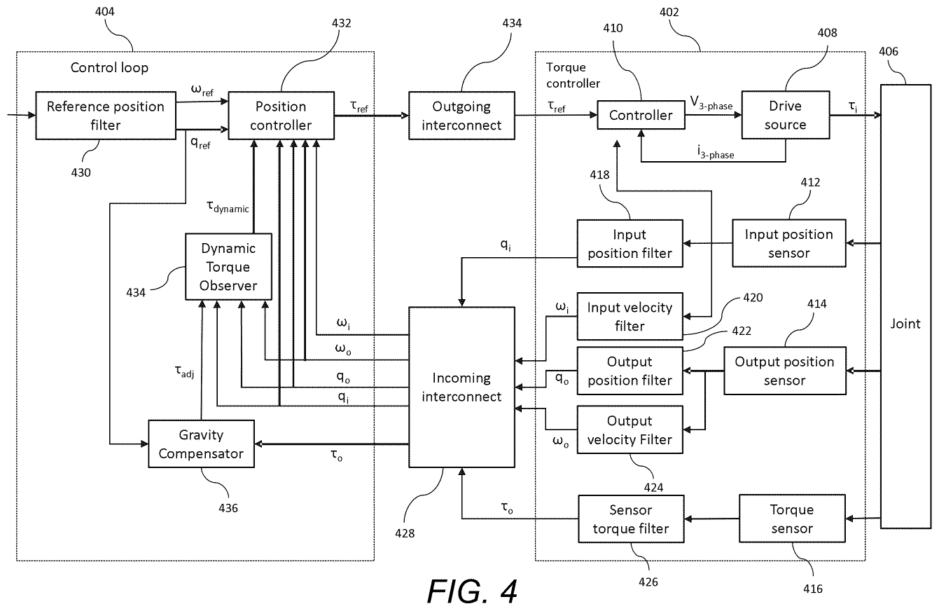

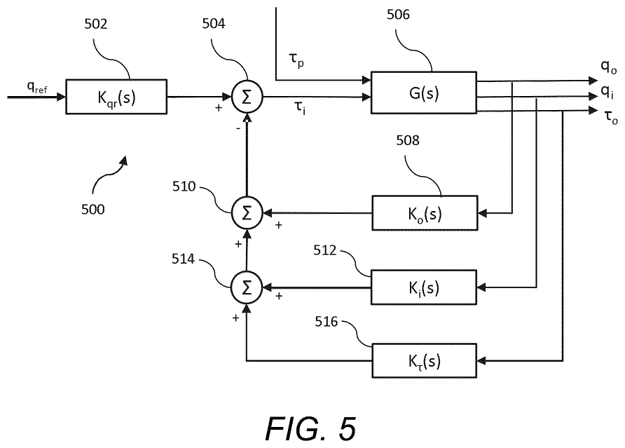

The output torque measured about the joint can be characterised by the following equation:

$${τ}_{o} = K ( {q}_{i} - {q}_{o} )$$

Where K is the spring coefficient, or stiffness, of the drivetrain, qᵢ is the input configuration of the joint and qₒ is the output configuration of the joint. In one example, as illustrated in FIGS. 4 to 5, qᵢ is measured at the first end of the drivetrain (proximal to the drive source) and qₒ is measured at the second end of the drivetrain (proximal to the joint). Thus, the output torque is characterised by a relationship between the elongation, (qᵢ−qₒ), and stiffness, K, of the drivetrain. In some examples, the relationship between elongation and stiffness K is linear. In other examples, this relationship is non-linear.

It is noted that the spring coefficient does not necessarily have a constant value for all elongation values. Indeed, in some examples, the spring coefficient is a continuous function of the elongation value, and in some other examples the spring coefficient is selected from a set of discrete values, with each discrete value being associated with a respective range of elongation values.

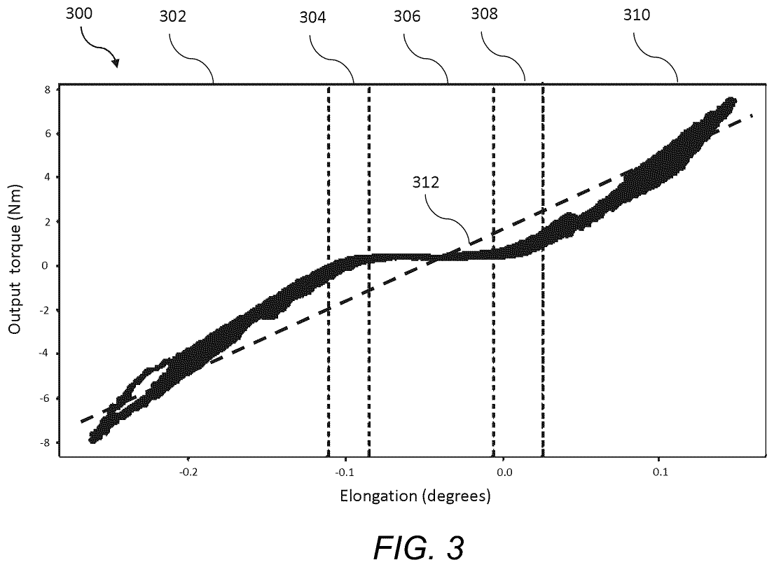

As described above, mechanisms for driving joints within a robotic system comprise an associated element of elasticity, which results in a non-linear stiffness characteristic. This elasticity is especially characteristic of harmonic drives. FIG. 3 is a graph illustrating an exemplary non-linear stiffness characteristic for an elastic joint in a robot arm. The graph plots elongation on the x-axis and output torque on the y-axis. The output torque may be measured by a sensor located at a second end of the drivetrain, at which the joint is located. The sensor for measuring output torque may be a gauge sensor. A gauge sensor is configured to measure changes in voltage due to mechanical strain. Elongation is calculated by subtracting the input configuration qᵢ from the output configuration qₒ of the joint. In FIGS. 4 to 5, the input configuration is measured by a first sensor located at a first end of the drivetrain at which the drive source is located. The output configuration is measured by a second sensor located at a second end of the drivetrain at which the joint is located.

In FIG. 3, output torque τₒ is measured in Nm and elongation is measured in degrees. In an alternative example, elongation may be measured in radians. In both of these examples, the first and second sensors for measuring the input and output configurations are rotary position sensors. In a further example, elongation may be a linear measurement that may be calculated using measurements obtained from linear position sensors. In this example, elongation may be measured in metres. In a further example, elongation may be dimensionless. An example of a dimensionless measurement of elongation is a percentage elongation.

The graph illustrated in FIG. 3 comprises five distinct regions 302-310. Regions 302 and 310 demonstrate a directly proportional relationship between drivetrain elongation and output torque τₒ. That is, as the elongation of the drivetrain increases, the output torque increases at a constant rate. This represents the idealised performance of the joint as described above with reference to FIG. 2. Region 302 illustrates joint performance for negative values of elongation, and region 310 illustrates joint performance for positive values of elongation. The gradient of the graph plotted in regions 302 and 310 represents the stiffness K of the drivetrain within the associated range of elongation values.

Region 306 represents the phenomenon of backlash, where the gradient of the line representing the stiffness of the drivetrain is near zero. As described above, backlash refers to the lost motion in a mechanism that is experienced when the mechanism changes direction. Such backlash is caused by gaps or clearance between interfacing components of the drivetrain. For a harmonic drive, backlash can result from the clearance between the circular spline and the flex spline, and/or from elastic deformation of the flex spline under torque. This phenomenon typically occurs within a particular region of elongation values. In the example illustrated in FIG. 3, this region of elongation values is between −0.1 and 0 degrees. It will be appreciated that, for different joints, the backlash region will present itself at a different range of elongation values. In this region of the graph, the measured sensor torque is zero and remains at zero as the elongation changes between −0.1 and 0 degrees. Thus, during the reversal of the joint direction between −0.1 and 0 degrees of elongation, no torque is transmitted through the drivetrain from the drive source to the joint.

Regions 304 and 308 are transition zones. In these zones the drivetrain is transitioning between the backlash region 306 and the regions of intended performance 302, 310. In the transition zones 304, 308, there is a non-linear relationship between elongation and torque.

The representative stiffness of the drivetrain throughout the range of elongation values recorded in FIG. 3 is represented by the gradient of a regression line 312 that passes through all regions of the graph. It is appreciated from FIG. 3 that, for an elastic joint, the relationship between elongation and output torque is not entirely linear. That is, the gradient of the stiffness characteristic in the backlash region 306 (which is zero or near zero) differs substantially from the gradient of the stiffness characteristic in regions 302 and 310. Thus, the straight line represented by 312 does not provide an accurate representation of the stiffness characteristic of the drivetrain for all values of elongation, but approximates the overall behaviour.

The performance of joints demonstrating non-linear characteristics as illustrated in FIG. 3 is difficult to accurately model. It is therefore useful for robotic control systems to incorporate feedback loops that measure the real-world performance of the joint and use those performance measurements as feedback terms to adjust the reference signals that are used to drive the joint.

The differential equations for a single elastic joint may be expressed as follows:

$$M {\ddot{q}}_{o} + D ( {\dot{q}}_{o} - {\dot{q}}_{ι} ) + K ( {q}_{o} - {q}_{i} ) + {F}_{o} = 0$$

$$B {\ddot{q}}_{ι} + D ( {\dot{q}}_{ι} - {\dot{q}}_{o} ) + K ( {q}_{i} - {q}_{o} ) + {F}_{i} = {τ}_{o}$$

In the above equations, M, B, K, D, Fᵢ and Fₒ are control variables. M is the inertia at the output of the drivetrain, B is the inertia at its input (i.e. at the output of the drive source), K is the spring coefficient for the drivetrain, and D is a damping term. D, therefore, represents the energy lost due to the elasticity in the drivetrain. Fᵢ is the input torque disturbance. Input torque disturbance may be due to static friction, for example. Fₒ is the output torque disturbance. In one example, output torque disturbance is caused by the other joints in the robot arm.

In addition to the abovementioned control variables, the differential equations observe four state variables for an elastic joint: position, velocity, acceleration and torque. These variables may be defined at either the input or output of the drivetrain. In the above equations the output configuration of the joint qₒ is an output position of the joint. Thus, \dot{q}ₒ and \ddot{q}ₒ are the first and second derivatives of the output position, respectively, with respect to time. That is, \dot{q}ₒ and \ddot{q}ₒ are the velocity and acceleration, respectively, at the second end of the drivetrain. Correspondingly, qᵢ, \dot{q}ᵢ, and \ddot{q}ᵢ are the position, velocity and acceleration at the first end of the drivetrain. The differential equations above can be parameterised by measuring the state variables described above using position and torque sensors located at or towards the first and second ends of the drivetrain. Velocity and acceleration are derivatives of position with respect to time, and so can be calculated from position measurements measured from position sensors. Torque data values are traditionally obtained from torque sensor measurements.

FIG. 4 is a block diagram illustrating a full state control scheme for controlling the configuration of a joint in a robot arm. In the specific example illustrated in FIG. 4, the configuration of the joint is its position. The control scheme may be comprised within the joint controllers described with reference to FIG. 1. The control scheme comprises a torque controller 402 and a control loop 404 that are electrically connected to each other by interconnect circuitry. The joint controller 402 comprises one or more electrical connections for connecting to the joint 406 of the robot arm. The robot arm may correspond to the arm 100 illustrated in FIG. 1, and the joint 406 may correspond to one or more of joints 106a-e.

The torque controller 402 is configured to calculate a value of input torque τᵢ to be generated by the drive source and transmitted by the drivetrain to the joint 406. The control loop 404 is configured to receive sensor data from the joint 406 and to calculate a reference torque value τ_{ref} using this sensor data as feedback.

The torque controller 402 comprises a drive source 408 and a drive source controller 410. The drive source 408 is connected to both the joint 406 of the robot arm and to the drive source controller 410. The drive source 408 is configured to supply an input torque τᵢ to the drivetrain that drives the joint 406 of the robot arm. In one example, the drive source 408 comprises a motor. In other examples the drive source 408 may comprise a hydraulic or pneumatic actuator, or any alternative power source. The drive source controller 410 is configured to control the performance of the drive source, and therefore to control the input torque about the joint 406.

The drive source 408 and the drive source controller 410 are electrically connected to each other by means of a feed forward loop passing from the controller to the drive source, and a feedback loop passing from the drive source to the controller. In an example where the drive source comprises a motor, the feed forward loop enables the controller to provide a three-phase voltage supply to the motor. In this example, the feedback loop enables the motor to supply a three-phase current feedback to the controller. The feedback loop is used to modify the voltage, and therefore the current, that is supplied to the motor. Thus, the feedback loop ensures that the intended torque is being generated by the motor.

The torque controller 402 further comprises a plurality of sensors for measuring the one or more parameters indicating the performance of the joint 406. The sensors are further configured to generate electrical signals associated with their measurements, such that these electrical signals can be transmitted to the control loop 404. In one example, the sensors are comprised within the joint 406. In another embodiment the sensors are external, but coupled, to the joint 406. The plurality of sensors may correspond to the sensors 116 illustrated in FIG. 1. In FIG. 4, the plurality of sensors comprises a first sensor 412, a second sensor 414 and a third sensor 416.

The first sensor 412 and the second sensor 114 are configured to measure the configuration about the joint 406. As mentioned above, the configuration of the joint may be the position of the joint. In this example, the sensors comprise encoders. The encoders may comprise linear encoders or rotary encoders, depending on the motion of the joint to which they are coupled. The first sensor 412 is configured to measure the input configuration of the joint 406. The input position is measured at the output of the drive source that is configured to drive that joint. Thus, the first sensor 412 may be located at, or at a position proximal to, a first end of the drivetrain, at which the drive source 408 is located. The first sensor 412 suitably comprises a position sensor. The second sensor 414 is configured to measure the output configuration at the joint 406. The output configuration is measured at the input to the joint. Thus, the second sensor is located at, or at a position proximal to, a second end of the drivetrain, at which the joint 406 is located. The second sensor 414 suitably comprises a position sensor. The third sensor 416 is configured to measure the output torque about the joint 406. The third sensor is suitably located at, or at a position proximal to, the second end of the drivetrain. The third sensor 416 suitably comprises a torque sensor. The first, second and third sensors 412, 414, 416 may be configured to continuously sense data indicating the performance of the joint. The first, second and third sensors 412, 414, 416 may be configured to periodically sense data indicating the performance of the joint. The periodic sensing is suitably performed at regular intervals. The sensing of data from the joint, e.g. using one or more of the first, second and third sensors, may be performed by the sensors at a predefined sampling rate. This sampling rate may be configurable, such as at the command interface 124 illustrated in FIG. 1. Suitably, the performance of the joint is sensed at least once in each 10 ms period. Thus, the sampling rate is suitably at least 100 Hz. Typically, sampling rates will be at least several hundred Hz, for example at least 1 kHz.

In addition to the sensors, the torque controller 402 comprises one or more sensor filters for filtering electrical signals received from a respective sensor. Filters are provided to combine information from multiple data values to efficiently monitor the performance of the joint. In one example, the filters are temporal filters, which are used to incrementally update their information for each new data value. The first filter 418 is configured to filter electrical signals received from the first sensor 412. Where the first sensor 412 is an input position sensor, the first filter 418 is configured to filter input position values. Correspondingly, a second filter 422 is electrically connected to the second sensor 414 and is configured to filter electrical signals received from that sensor. Where the second sensor 414 is an output position sensor, the second filter 422 is configured to filter output position values. A third filter 426 is electrically connected to the third sensor 416. Where the third sensor 416 is a torque sensor, the third filter 426 is suitably configured to filter output torque values.

The torque controller 402 further comprises fourth and fifth filters 420, 424 for deriving filtered derivative values from the first and second sensors 412, 414 respectively. As mentioned above, the sensing of data by the sensors 412, 414, 416 may be performed at a predefined sampling rate. The fourth and fifth filters 420, 422 are configured to maintain a record of the sampling rate at which sensor data is measured. Velocity is the first derivative of position with respect to time. Thus, where the first and second sensors 412, 414 are input and output position sensors, the fourth and fifth filters 420, 422 are configured to calculate input and output velocity values for the joint, respectively, using the predefined sampling rate and the sampled data values.

As well as being electrically connected to the one or more sensors, the one or more sensor filters are electrically connected to incoming interconnect circuitry 428. The incoming interconnect circuitry 428 is further connected to the control loop 404. The interconnect circuitry 428 is therefore configured to transmit sensor data from the torque controller 402 to the control loop 404. Thus, the sensor data may be provided as feedback to the control loop 404. In one example, the interconnect circuitry is a communication bus, such as an Ethercat Bus.

The control loop 404 is configured to calculate a reference torque τ_{ref} to be provided to the drive source 408. The reference torque τ_{ref} can be calculated using a reference configuration q_{ref} and a plurality of feedback loops derived from measured sensor data. The control loop 404 comprises a sixth filter 430, a configuration controller 432, a dynamic torque observer 434 and a gravity compensator 436.

The sixth filter 430 is connected to the configuration/position controller 432 and is configured to receive one or more incoming reference configuration value q_{ref} (such as from control unit 118). The sixth filter 430 is further configured to filter these values to provide an input to the configuration controller 432. The filter 430 is also configured to calculate a value for the first derivative of this reference configuration q_{ref}. Where the reference configuration is a reference position for the joint 406, the first derivative of this reference configuration (with respect to time) is reference velocity. The sixth filter 430 may correspond to the filters 418, 420, 422, 424, 426 of the torque controller 402. That is, the position filter may be configured to combine information from multiple data values to efficiently monitor the input position values, and to calculate a reference velocity value using a stored predefined sampling rate and the continuously sampled reference position values.

The control loop further comprises a gravity compensator 436, which is electrically connected to both the output of the sixth filter 430 and the incoming interconnect circuitry 428. The gravity compensator 436 is configured to receive reference configuration data from the sixth filter 430 and output torque values from the third sensor 416 by means of the interconnect circuitry 428. The gravity compensator 436 is further configured to calculate an expected torque value using the data obtained from the sixth filter 430. A value of the gravity-compensated measured torque can then be calculated by the gravity compensator 436 by subtracting expected torque from output torque τₒ. This gravity-compensated measured torque is provided to dynamic torque observer 434.

In addition to the gravity-compensated measured torque, the dynamic torque observer 434 is configured to receive values of input configuration, output configuration and the first derivative of output configuration from the interconnect circuitry 428. The dynamic torque observer 434 is further configured to calculate a dynamic torque value τ_{dynamic} from its received inputs. The dynamic torque value τ_{dynamic} is transmitted by the dynamic torque observer 434 to the configuration controller 432. The configuration controller 432 is also electrically connected to the sixth filter 430 and interconnect circuitry 428. The configuration controller 432 is therefore configured to receive, in addition to τ_{dynamic}, the input and output configurations, and the first derivatives of these configurations, from the incoming interconnect circuitry 428. The position controller is further configured to receive the reference configuration and first derivative of this value from the sixth filter 430. From these received values, the configuration controller 432 calculates a value for the reference torque τ_{ref} to be provided to the torque controller 402. The reference torque τ_{ref} is transmitted from the command loop 404 to the torque controller 402 by outgoing interconnect circuitry 434. As with incoming interconnect circuitry 428, outgoing interconnect circuitry 434 may be a communication bus, such as an Ethercat bus.

FIG. 5 is a block diagram illustrating a control algorithm 500 for controlling the configuration of a robotic joint that uses torque sensor data as a feedback term to calculate a value of input torque τᵢ. The position control algorithm may be comprised within the configuration controller 432 illustrated in FIG. 4, for example. The position control algorithm uses a reference configuration q_{ref} (received from control unit 118, for example) and a plurality of feedback terms to calculate an input torque value Ti to be provided to a drive source. The reference configuration q_{ref} is initially multiplied by a first transfer function 502 and is provided to first summing point 504. The first transfer function 502 comprises a first gain that is provided to manipulate the raw input value of q_{ref}.

The control algorithm 500 further comprises a second transfer function 506 which comprises a gain G(s) that is associated with the drive source. In an example where the drive source is a motor, the gain G(s) comprises the motor gain. The value of motor gain G(s) is dependent on the physics of the joint. A value of physical torque τₚ and input torque τᵢ are provided as inputs to the second transfer function 506. The physical torque τₚ that is applied to the second transfer function 506 represents the real-life disturbance to the joint system. This torque does not exist electronically. The outputs of the second transfer function 506 are output position qₒ, input position qᵢ and output torque τₒ. These outputs are measured parameters that are obtained from sensors located at the joint and its respective drivetrain. The sensors may, for example, correspond to sensors 412, 414, 416 as illustrated in FIG. 4.

The three outputs qₒ, qᵢ, τₒ from transfer function 506 that are measured at the joint are provided as feedback terms that are used to calculate the input torque τᵢ. Each measured parameter is provided with an associated feedback loop. Each feedback loop comprises a transfer function that corresponds to the output value with which it is associated. For example, the feedback loop for measured output configuration qₒ comprises a third transfer function 508. In the example illustrated in FIG. 5 the third transfer function comprises one associated gain Kₒ. In an alternative example, the third transfer function 508 comprises more than one gain. The third transfer function 508 is configured to calculate manipulated values of both the output configuration and the first derivative of output configuration \dot{q}ₒ to provide to summing point 510. In the example in which the transfer function comprises more than one gain, a gain may be provided for each derivative of the output configuration provided as an input to the transfer function 508. So, where the third transfer function is configured to calculate manipulated values of both the output configuration and the first derivative of the output configuration, the third transfer function 508 may comprise two gains. In a further alternative example, the third transfer function 508 may comprise a matrix, where each element of the matrix is a function relating a particular input variable to an output variable.

The feedback loop provided for measured input configuration qᵢ comprises a fourth transfer function 512. Similarly to the third transfer function 508, in the example illustrated in FIG. 5, the fourth transfer function comprises one associated gain Kᵢ. In an alternative example, the fourth transfer function 512 comprises more than one gain. Specifically, where the fourth transfer function is configured to calculate manipulated values of both the input configuration and the first derivative of the input configuration, the fourth transfer function 512 may comprise two gains. In a further alternative example, the fourth transfer function 512 may comprise a matrix, where each element of the matrix is a transfer function relating a particular input variable to an output variable. The fourth transfer function 512 is configured to calculate manipulated values of both the input configuration and the first derivative of input configuration 4, to provide to summing point 514.

The feedback loop provided for output torque τₒ comprises a fifth transfer function 516 with an associated gain K_{τ}. A manipulated value of output torque τₒ is thus provided to summing point 514. The gains Kₒ, Kᵢ, K_{τ} may be predefined continuous or constant values that may be obtained from predetermined theoretical or experimental data. The gains may be stored in a non-transient form within the control system for the robot arm. The gains may be applied to the physical measurement data and are used to affect the performance of the control algorithm.

The feedback loops are summed together at summing points 510, 514, and are used to calculate the input torque τᵢ at summing point 504. That is, summing point 504 receives manipulated reference configuration q_{ref} and the manipulated sensor data feedback as inputs, and outputs input torque τᵢ. Knowing the configuration of the joint at the input and at the output of the drivetrain allows the control system to determine the elongation of the drivetrain. Thus, the joint can be controlled in dependence on that elongation.

Using the control algorithm illustrated in FIG. 5, the value of input torque τᵢ calculated by the controller is represented by the following equation:

$${τ}_{i} = {k}_{po} ( {q}_{ref} - {q}_{o} ) + {k}_{pi} ( {q}_{ref} - {q}_{i} ) + {k}_{do} ( {\dot{q}}_{ref} - {\dot{q}}_{o} ) + {k}_{di} ( {\dot{q}}_{ref} - {\dot{q}}_{i} ) - {K}_{t} {τ}_{o}$$

In the above equation, q_{ref} is a reference configuration that is computed for each joint of the arm. The configuration may be angular or linear, depending on the motion of each joint. kₚₒ, kₚᵢ, k_{do} and k_{di} are gains for the first and second inputs. kₚₒ and kₚᵢ are proportional gains for the input and output configurations respectively. k_{do} and k_{di} are derivative gains for the input and output configurations respectively. The first derivative of reference and measured configurations \dot{q}_{ref}, \dot{q}ᵢ and \dot{q}ₒ are synonymous with the respective velocities ω_{ref}, ωᵢ and ωₒ.

The control system illustrated in FIG. 5 requires five measured feedback terms (qᵢ, qₒ, \dot{q}ᵢ, \dot{q}ₒ, τₒ) to calculate input torque τᵢ. However, measured output torque τₒ cannot always be used to provide an accurate representation of the performance of a joint. This is of particular relevance to joints with drivetrains which experience backlash, such as harmonic drives. Generally, the elongation of the drivetrain varies linearly with variation in the torque applied to that joint. As described above, in the backlash region of a non-linear joint the measured sensor torque is near zero as the joint elongation varies. Thus, in the backlash region, torque sensors cannot reliably be used to provide values of measured torque.

As introduced above, the output torque for a robotic joint can be characterised by the following equation:

$${τ}_{o} = K ( {q}_{i} - {q}_{o} )$$

Where K is the spring coefficient of the joint, qᵢ is the input configuration about the joint and qₒ is the output configuration about the joint. As previously mentioned, qᵢ−qₒ represents the elongation of the drivetrain. qᵢ and qₒ can both be measured using sensors coupled to the joint and its associated drivetrain.

### Configuration Error

The difference between a joint input position (qᵢ) and a joint output position (qₒ) can, as described herein, be termed an elongation. This is an amount by which the drive mechanism, e.g. the drivetrain, elongates due to its elasticity. This difference, qᵢ−qₒ, can also be termed a configuration error, or tracking error, as it provides a measure of the error in an actual configuration compared to a desired configuration.

In a perfectly “stiff” drive mechanism, the presence of a configuration error is indicative of a fault in the system, since there should be no reason for the input position and output position to differ from one another. Thus, in this situation, a system fault can be raised where there is a configuration error (perhaps limiting the system fault conditions to an error that exceeds a tolerance level, to account for measurement errors and the like).

In a real drive mechanism that is not perfectly “stiff” but has some inherent elasticity, such as the drive mechanism described herein, the presence of a configuration error is not necessarily indicative of a fault in the system. For a drive mechanism that has some elasticity, the greater the torque applied, the greater the configuration error is likely to be (this follows from the equation above). Where the mismatch between the input position and the output position is not excessive, the drive mechanism and joint can continue to operate normally (that is, within normal or accepted bounds of operation) despite there being a configuration error.

However, as with the “stiff” drive mechanism, there will be a value for the configuration error that indicates a fault in the system. The fault could, for example, be that gear slip has occurred (in a drive mechanism that comprises gears), that a component in the drive mechanism is damaged, that a component in the drive mechanism is approaching or has exceeded a plastic deformation limit, that physical damage to one or more sensor has occurred, that there is an error such as a software error in handling the sensor data, and so on. Such faults can represent critical damage to the system (such as the breakage of a component that then needs replacing). The fault need not be a serious fault. For example, the fault can indicate that, whilst safe operation can continue, the system is approaching a position in which a more serious fault such as component damage might occur. This type of fault can indicate to a user of the robotic system that action is required to avoid the more serious fault occurring.

As described elsewhere herein, the input position and the output position can deviate from one another approximately linearly as increasing torque is applied to the joint, complying with an “open-loop stiffness” law.

Thus, it is desirable to monitor for the occurrence of (i) configurations where damage is likely as well as (ii) configurations where damage is unlikely to occur but corrective action is advised to avoid entering a configuration where damage is likely to occur.

A configuration error limit can be set, for example in system software, that can indicate a limit to an acceptable configuration error for a joint. This configuration error limit is suitably joint-specific. This permits the characteristic of the joint to be taken into account when determining the appropriate configuration error limit for that joint. For example, a relatively stiffer joint can have a configuration error limit that is lower than for a relative less stiff joint. This is because the relatively less stiff joint can be permitted to elongate more under a given torque without that elongation being indicative of damage. Joints can have different stiffnesses for several reasons. Joints differ in size and construction, and so will have different stiffnesses. The different physical arrangements of joints can result in different stiffnesses. Different materials, or types of materials (or even different batches of the same type of material) can result in different stiffnesses. Different manufacturing techniques can result in joints having different stiffnesses.

A configuration error limit can be set for the controller. This configuration error limit can be set at a value that is indicative of a serious fault arising. Thus, where the configuration error reaches or exceeds this configuration error limit, the system can enter a fault state. On entering this fault state, the system can suspend operation of the robot arm by the user. The system can prevent or restrict movement that would result in the configuration error increasing. The system may permit movement that would result in the configuration error decreasing. In some implementations, the system can, on entering the fault state, decouple a user input device used for controlling movement of the robot arm such that the robot arm is not operatively coupled to the user input device. This means that the user is not able to further manipulate the robot arm. This response to entering the fault state prevents any further possible damage occurring to the robot arm. Where damage may already have occurred to the robot arm, this response to the fault state prevents further operation of the robot arm by the user. Where damage has occurred, the manipulation of the arm by the user may differ from expected operational control. Thus, restricting further manipulation by the user avoids incorrect or unintended manipulation of the arm. Such unintended manipulation of the robot arm can be undesirable in factory settings, and can be critical in surgical robotic settings and in other safety-critical processes.

Where the fault state is entered, and the response of the system is to operationally decouple the robot arm from the user input device, the system can be configured not to permit re-coupling of the user input device to the robot arm. Thus, in the surgical robotic system described herein, the system user can be forced to manually remove an instrument from a surgical site and to remove that instrument from the robot arm. This can be done manually in a safe way. A replacement instrument and/or a replacement robot arm can be used to continue with the robotic procedure. Whilst this response maintains safety of operation, it is cumbersome for the system operator and surgical team, and can introduce undesirable delays into the surgical procedure.

Carrying out this process is necessary where the configuration error indicates possible damage to the system. However, there are cases where this instrument removal process is not necessary. Consider two joints of the robot arm that have different inherent stiffnesses. The more flexible joint can operate within its normal operating range with a configuration error up to a particular value, CE1. The stiffer joint can operate within its normal operating range with a configuration error up to a different particular value, CE2. CE2 will be lower than CE1. Where the configuration error limit is set ‘globally’, such that it applies to both the more flexible joint and the stiffer joint, it must be set at a level suitable for the stiffer joint. Thus, the configuration error limit is likely to be set at a level close to but lower than CE2. Since CE2 is lower than CE1, the configuration error limit will be well within the normal operating range of configuration errors of the more flexible joint.

Where the more flexible joint experiences relatively high torques, the configuration error may exceed the configuration error limit whilst remaining less than CE1. However, where there is a ‘global’ configuration error limit, the system will enter the fault state, thus necessitating the instrument removal process. In this case, that instrument removal process will not be needed. Hence, forcing this process to occur for the more flexible joint leads to unnecessary system delays and a burden on the surgical team.

It is therefore desirable to set configuration error limits that take into account the characteristics of the joint, such as joint stiffness.

In one implementation, the configuration error may be monitored by separate processes within the overall robotic system. This redundancy of checking is important in safety-critical scenarios such as robotic surgery. The configuration error can be monitored at the joint controller and at the robot arm controller. Since these controllers operate at different parts of the software control system, there may be different responses to fault conditions detected at each of these controllers. For example, where a fault condition is detected at the joint controller (e.g. the configuration error reaches or exceeds a joint controller configuration error limit) a relatively more serious fault can be detected, which can result in, for example, the above-described fault state being entered.

Where a fault condition is detected at the robot arm controller (e.g. the configuration error reaches or exceeds a robot arm controller configuration error limit) a relatively less serious fault can be detected, which can result in an alternative fault state being entered. This alternative fault state may comprise providing an indication to the user that the fault has occurred, such as by an audio and/or visual indication, and/or in another suitable way. This might include haptic feedback for example. In this alternative fault state, operational control of the robot arm by the user may be permitted to continue. In a different implementation, in the alternative fault state, the user input device can be decoupled from the robot arm such that the user input device is not operationally coupled to the robot arm. However, in contrast to the system response in the fault state, in the alternative fault state, the system may permit re-coupling of the user input device to the robot arm. For example, the user may be able to re-enable operational control after satisfying safety checks that are less stringent than those of the fault state.

It can therefore be appropriate to set different configuration error limits for detection by different parts of the control system, to improve reliability of robotic control effected by the control system. For example, the robot arm controller configuration limit can be set at a lower level than the joint controller configuration error limit. This approach helps to ensure that faults are caught by the robot arm controller before they are caught by the joint controller. That is, the setting of different configuration error limits for comparing to the configuration error at different parts of the control system can help ensure that a less serious response is caused by the configuration error meeting or exceeding a first, lower, limit, whilst the more serious response is still caused by the configuration error meeting or exceeding a second, higher, limit.

Thus, the system can be configured to detect a class of faults (e.g. by the robot arm controller) that are less serious than a different class of faults (e.g. those detected by the joint controller), and to respond differently, depending on the class of the fault detected. This can improve the operation of the robotic system, reducing potential delays, whilst maintaining safety and reliability of operation.

Suitably, there are therefore joint-specific configuration error limits and/or controller-specific configuration error limits. The use of such configuration error limits, separately and/or together, improves the operational capability of the robotic system.

It is noted that it is important to check that the mismatch between the input position and the output position corresponds to the mismatch (or configuration error) that would be expected for a given load or torque applied to the joint. That means that the direction of the torque being applied, and the direction of the mismatch also needs to match.

There is provided a controller for controlling the configuration of a joint in a surgical robot. The joint is driven by a drivetrain which transfers power from a drive source to the joint. The controller is configured to receive a drive source configuration signal indicating a configuration of the drive source. The drive source configuration signal can comprise or indicate the input position. The controller is configured to receive a joint configuration signal indicating a configuration of the joint. The joint configuration signal can comprise or indicate the output position.

The controller is configured to determine a configuration error from a difference in configurations indicated by the drive source configuration signal and the joint configuration signal. For example, the drive source configuration signal may indicate, in the joint frame of reference, that the input position is, say, a rotational position of 60 degrees. The joint configuration signal may indicate, also in the joint frame of reference, that the output position is 61.4 degrees. Thus, the configuration error can be found from the difference between these two values, i.e. taking the difference as qᵢ−qₒ, the configuration error is 60-61.4 degrees, or −1.4 degrees.

It will be understood that this difference gives a sense of the ‘direction’ of the error, in that the output position may be either greater than the input position or less than the input position. (Where the output position equals the input position, the configuration error is zero.)

The controller is configured to determine a configuration error limit from a measured joint torque of the joint and a joint stiffness of the joint. Suitably, the joint stiffness of the joint in this determination is a minimum joint stiffness, Kₘᵢₙ. Rearranging the relationship above gives:

$$( configuration error ) = T / {K}_{min}$$

It can therefore be seen that, for a given stiffness, the configuration error is permitted to increase with torque. A smaller minimum stiffness will lead to a larger permitted configuration error. Since there is a limit on the value of the configuration error that can be accepted, i.e. without leading to potential or actual damage occurring, there is a limit on the minimum stiffness value for this determination of the configuration error limit. That is, the minimum stiffness is selected such that the configuration error limit is set at a suitable level for the particular joint, and/or the particular use case.

Suitably the minimum stiffness is a predetermined value. The minimum stiffness can be determined from joint usage data. The minimum stiffness can be determined from telemetry data, such as telemetry data from one or more procedures carried out using the robot. The minimum stiffness can be determined by testing the joint, for example during one or more of manufacture, assembly, commissioning, during a test procedure, and the like. Suitably, the testing forms part of a manufacturing process of the joint, or of the arm incorporating the joint. Preferably, the minimum stiffness is a predetermined value stored in a manner that is accessible to the controller, for example by being stored in a memory accessible to the controller.

The controller is configured to compare the determined configuration error and the determined configuration error limit and, in dependence on the comparison, to output a fault signal where the determined configuration error equals or exceeds the determined configuration error limit.

The fault signal is indicative of a fault condition, for example that the configuration error is reaching a value at which damage may occur, or that the configuration error has reached a value at which damage is likely to occur.

Preferably the output position is a measured output position. That is, the joint configuration signal preferably comprises a measured joint configuration. The controller suitably comprises an output position sensor configured to sense the joint configuration. The input position may be a commanded position. The commanded position may be obtained from a drive controller for controlling the drive mechanism. Obtaining the input position in this way is useful as it can avoid the need to provide the sensor for sensing the input position. It also means that where a sensor for sensing the input position is provided, using the commanded input position can be used as a check on the input position. The input position may be a measured input position. For example, an input position sensor may be provided for sensing the drive source configuration.

Using the measured joint configuration signal and/or the measured drive configuration signal can help ensure that the actual (rather than a desired) status of the drive mechanism and the joint are determined. This can help improve the accuracy of the configuration error determination. Suitably the drive source configuration signal comprises an indication of the measured physical position of the drive source. Suitably the joint configuration signal comprises an indication of the measured physical position of the joint.

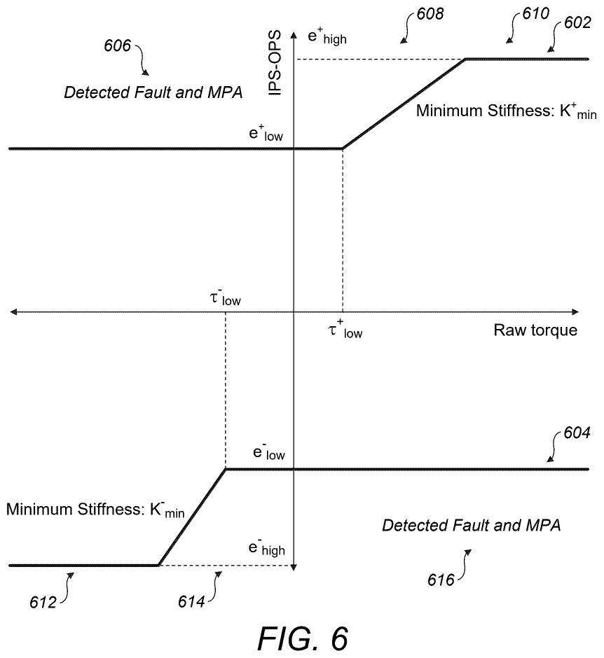

Further details of the fault detection scheme implemented by the controller will now be described with reference to FIG. 6. FIG. 6 shows a plot of the configuration error (input position (IPS)−output position (OPS)) against torque. There are fault thresholds indicated by the upper line 602 and the lower line 604. The upper line shows the fault thresholds for positive configuration errors. The lower line shows fault thresholds for negative configuration errors.

Each of the upper line 602 and the lower line 604 comprise three segments. The upper line 602 comprises a first segment 606 at relatively low torques, a second segment 608 at intermediate torques and a third segment 610 at relatively high torques. The first segment 606 shows a constant configuration error limit, e⁺_{low}, over a range of torques from low torque up to a τ⁺_{low} torque value. The τ⁺_{low} value, as illustrated in FIG. 6, is a small positive torque value. The second segment 608 shows a linear relationship between torque and configuration error for torques above the τ⁺_{low} value up to a predetermined value of the configuration error, e⁺_{high}. The gradient of the second segment 608 is indicative of a minimum stiffness value for positive configuration errors, K⁺ₘᵢₙ. The third segment 610 shows a constant configuration error of e⁺_{high}.

The lower line 604 comprises a fourth segment 612 at relatively low torques, a fifth segment 614 at intermediate torques and a sixth segment 616 at relatively high torques. The sixth segment 616 shows a constant configuration error limit, e⁻_{low}, over a range of torques from high torque down to a τ⁻_{low} torque value. The τ⁻_{low} value, as illustrated in FIG. 6, is a small negative torque value. The fifth segment 614 shows a linear relationship between torque and configuration error for torques less than τ⁻_{low} down to a predetermined value of the configuration error, e⁻_{high}. The gradient of the fifth segment 614 is indicative of a minimum stiffness value for negative configuration errors, K⁻ₘᵢₙ. The fourth segment 612 shows a constant configuration error of e⁻_{high}.

Preferably, for each joint with an output position sensor, the controller is configured to compare the input position with the output position, taking account of the torque applied to the joint. If the difference between the expected joint position calculated from the input position (IPS) and the actual joint position measured by the output position (OPS) exceeds (in positive or negative direction) the range given by the piece-wise linear maps Y⁻(T) and Y⁺(T) defined below and shown in FIG. 6, a fault can be raised by the controller. Table 1 shows example parameters for a robot arm with 8 joints.

$$τ < {Y}^{-} ( τ ) = \{ \begin{matrix}{e}_{high}^{-} & if {e}_{high}^{-} is specified , \\ {e}_{low}^{-} & τ > {τ}_{low}^{-} , \\ {e}_{low}^{-} + ( 1 / {K}_{min}^{-} ) ( τ - {τ}_{low}^{-} ) & τ \leq {τ}_{low}^{-} .\end{matrix}$$

$$τ > {Y}^{+} ( τ ) = \{ \begin{matrix}{e}_{high}^{+} & if {e}_{high}^{+} is specified , \\ {e}_{low}^{+} & τ < {τ}_{low}^{+} , \\ {e}_{low}^{+} + ( 1 / {K}_{min}^{+} ) ( τ - {τ}_{low}^{+} ) & τ \geq {τ}_{low}^{+} .\end{matrix}$$

TABLE 1
Example parameters for the piece-wise linear maps Y⁻(τ) and
Y⁺(τ) for a robot arm with 8 joints.

| | τ⁻_{low} | τ⁺_{low} | e⁻_{low} | e⁺_{low} | e⁻_{high} | e⁺_{high} | K⁻ₘᵢₙ | K⁺ₘᵢₙ |
|---|---|---|---|---|---|---|---|---|
| Joint | (Nm) | (Nm) | (deg) | (deg) | (deg) | (deg) | (Nm/deg) | (Nm/deg) |
| J1 | 0.0 | 0.0 | −0.5° | 0.5° | −2.5° | 2.5° | 49.7 | 49.7 |
| J2 | 25.0 | −25.0 | −0.5° | 0.5° | −2.5° | 2.5° | 37.5 | 37.5 |
| J3 | 10.0 | −10.0 | −0.5° | 0.5° | −2.5° | 2.5° | 34.9 | 34.9 |
| J4 | 15.0 | −15.0 | −0.5° | 0.5° | −2.5° | 2.5° | 31.4 | 31.4 |
| J5 | 5.0 | −5.0 | −0.5° | 0.5° | −2.5° | 2.5° | 6.1 | 6.1 |
| J6 | 2.5 | 0.0 | −2.0° | 2.0° | −5.0° | 5.0° | 0.8 | 1.3 |
| J7 | 5.0 | −5.0 | −2.0° | 2.0° | −5.0° | 5.0° | 1.3 | 1.3 |
| J8 | — | — | — | — | −5.0° | 5.0° | — | — |

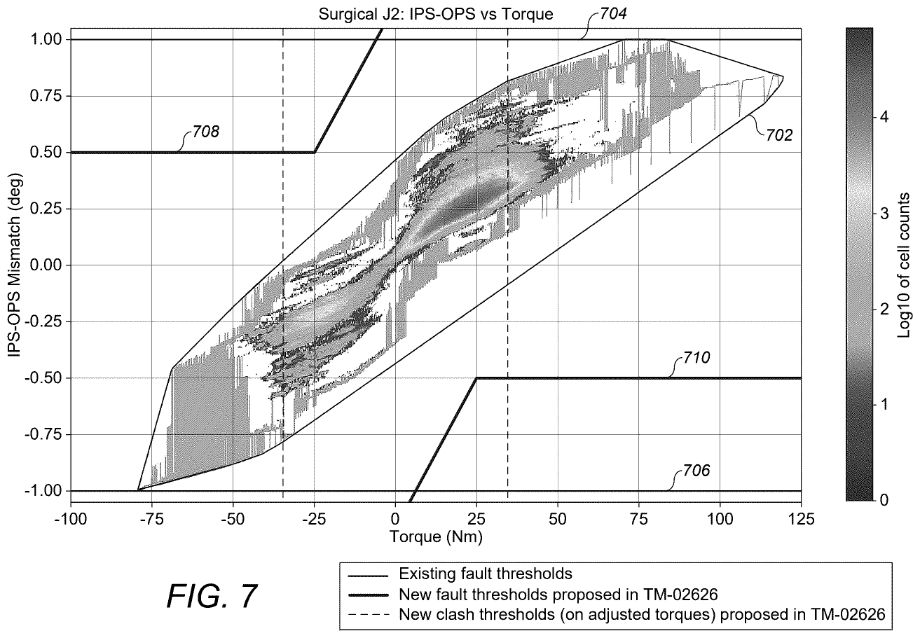

The configuration error scheme discussed herein can be seen in FIG. 7, together with an alternative configuration error scheme in which the configuration error limits are constant values for both positive and negative configuration errors for all torque values. FIG. 7 shows the stiffness relationship for a real joint in a robot arm, based on joint data captured during operation of the joint. It will be understood that the values indicated in FIG. 7 are specific to the particular joint and the particular operation of that joint. However, the principles illustrated in FIG. 7 are more generally applicable. FIG. 7 shows a graph of configuration error (IPS-OPS mismatch) against torque. The relationship between configuration error and torque for this joint reveals an approximately linear trend to the relationship (indicated at 702), whilst exhibiting the features discussed elsewhere herein in respect of FIG. 3.

Suitably, raw torques are used (rather than gravity-compensated torques) because the configuration error is directly related to raw torque in the open-loop joint stiffness model. Such a stiffness model is applicable to at least some joints of the robot arm.

The horizontal lines at positive configuration error 704 and at negative configuration error 706 indicate simple configuration error limits that do not take into account the stiffness of the joint. Taking the upper line 704, at positive configuration error, it can be seen that at torques lower than approximately 25 Nm there is a gap between the joint configuration error data 702 and the upper line 704. This gap widens as torque decreases. Thus, close to torque values of approximately 25 Nm, the simple configuration error limit shown by the upper line 704 provides an appropriate configuration error limit that permits operation of the joint within normal operating parameters, whilst providing a limit past which it is appropriate to indicate that a fault has arisen. However, at increasingly lower torques, the widening gap between the joint configuration error data 702 and the upper line 704 means that increasingly large deviations from normal operating parameters (i.e. larger than usual configuration errors) are needed to trigger the fault. Thus, it is possible that undesirably high configuration errors might occur before the fault is triggered. This makes it more likely that a more serious fault will be triggered, causing delays to the operation of the robotic system.

At torques higher than approximately 25 Nm, the joint configuration error data 702 approaches close to the configuration error limit provided by the upper line 704. At torques in the region of 75 Nm, configuration errors that are typical of normal operation of the joint can be seen to reach the configuration error limit provided by the upper line 704. This means that the fault will be triggered even during normal operation of the joint when the joint operating parameters are within normal bounds. Such a fault is an example of a ‘false positive’ fault. That is, the fault triggers unnecessarily. The system response to such a fault would therefore cause unnecessary delays to the operation of the robotic system.

As can be seen in FIG. 7, a similar relationship is true for negative configuration errors and the lower line 706.

The simple configuration error limits provided by the upper line 704 and the lower line 706 of FIG. 7 therefore provide a sub-optimal way of detecting configuration error faults. In contrast, the present configuration error limit scheme is indicated at lines 708 and 710. Only part of the configuration error limits illustrated in FIG. 6 are shown in FIG. 7. It can be seen, from a comparison of the upper line 704 and line 708, against the joint configuration error data 702, that line 708 provides a much tighter configuration error limit at lower torque magnitudes (i.e. closer to zero). At lower torques, below −25 Nm in FIG. 7, the positive configuration error limit 708 is a constant. This means that configuration error checks are made on the input position and output position difference alone. The positive configuration error limit 708 is closer to the normal range of expected configuration errors than the simple configuration error limit provided by the upper line 704. At torques higher than 25 Nm (as illustrated) the allowed configuration error is a linear function of the torque applied, matching the joint stiffness characteristics, up to a maximum allowed error. The joint stiffness is such that the new wider maximum configuration error discrepancy is still well within the joint overload capacity. Thus, at such higher torques, the input position and output position difference is expected to grow with increasing raw torque, whilst maintaining a minimum mechanical stiffness. A maximum allowed configuration error is specified (e⁻_{high} and e⁺_{high}, see FIG. 6). This maximum allowed configuration error can be used by the robot arm controller. The configuration error limit used by the joint controller can be a larger configuration error than this robot arm controller maximum allowed configuration error. This can help ensure that the robot arm controller configuration error limit is within the joint controller configuration error limit. Thus, where the robot arm controller configuration error limit is reached or exceeded, without reaching the joint controller configuration error limit, a less serious fault can be raised by the system, which can permit a quicker and/or easier system re-start. If a determined configuration error meets or exceeds the relatively wider joint controller configuration error limit, it is more likely that this fault will indicate electro-mechanical damage to the joint.

In some examples, the joint controller configuration error limit exceeds the robot arm controller configuration error limit by a degrees. Suitably a is at least 0.5 degrees. a may be 1 degree or greater, for example 1.5 degrees or 2 degrees. Suitably a is less than or equal to 3 degrees, for example 2 degrees. This range of values for a helps ensure that the more serious fault of meeting or exceeding the joint controller configuration error limit is not triggered unnecessarily, but that reliable and safe operation of the surgical robot is maintained at all times.

Tightening the checks for certain joints at low torques can reduce risk to a patient undergoing a surgical procedure using a surgical robot, because lower deviation from normal operating parameters (i.e. from a range of configuration errors typically experienced in normal operation) is accepted before a fault is raised.

The portion of the configuration error limit of line 708 showing the linear relationship between configuration error and torque enables a check to be made to confirm that the input position and output position are consistent and that the joint does not demonstrate a reduction in mechanical joint stiffness below a minimum stiffness. This check can help confirm that the level of configuration error does not indicate damage to the joint or failure of the software control.

In some examples, an additional torque threshold can be provided. The additional torque threshold can warn users of the robot about a potential gear-slip in a geared drive mechanism. Gear-slip can occur when large torques are applied in the negative torque direction. The additional torque threshold is a torque threshold designed to warn the user about a large torque being applied to the joint. This additional torque threshold is useful because, even if the stiffness threshold has not been violated, it would be easier for gears to slip under high torques. The additional torque threshold is suitably set at a torque level lower than that at which gear-slip is expected to occur, for example within about 30 Nm of such a gear-slip torque, or within about 20 Nm of such a gear-slip torque, or within about 10 Nm of such a gear-slip torque, or within about 5 Nm of such a gear-slip torque. Thus, the user can be warned in advance of the gear-slip occurring, enabling them to adjust control of the robot arm to avoid gear-slip. In response to the torque meeting or exceeding the additional torque threshold, an audio and/or visual signal can be generated.

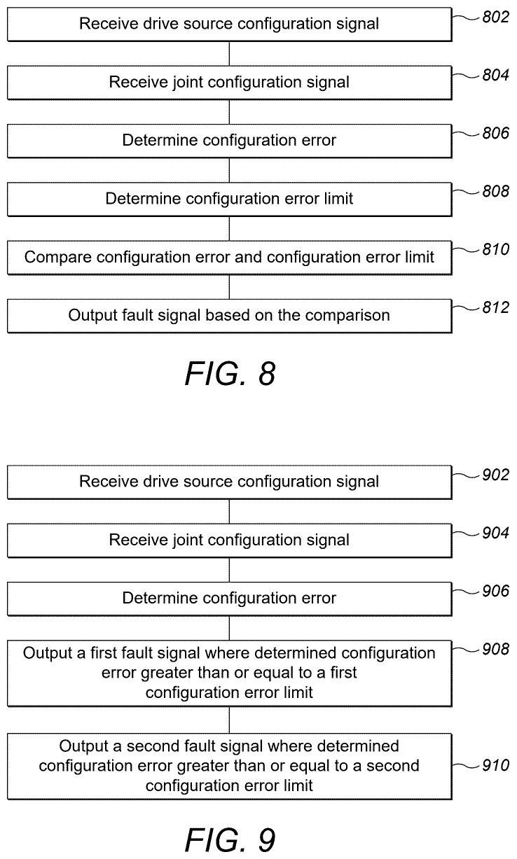

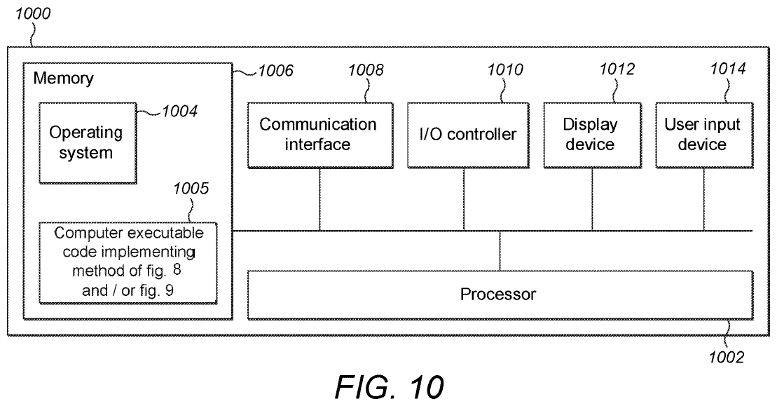

A method of controlling the configuration of a joint in a surgical robot will now be described with reference to FIG. 8. At 802, a drive source configuration signal is received, for example at a controller. The drive source configuration signal is indicative of the configuration of the drive source. For example, the drive source configuration signal can indicate a position of the drive source. The drive source configuration signal can comprise a measure of the position of the drive source. A joint configuration signal is received, at 804. The joint configuration signal is suitably received at the controller. The joint configuration signal is indicative of the configuration of the joint. For example, the joint configuration signal can indicate a position of the joint. The joint configuration signal can comprise a measure of the position of the joint.

A configuration error is determined 806 from the difference between the configurations indicated in the drive source configuration signal and in the joint configuration signal. Suitably, the configuration error is determined from the difference in the position determined from the drive source and the position determined from the joint.

A configuration error limit is determined 808 based on a measured joint torque of the joint and a joint stiffness of the joint. The configuration error and the configuration error limit are compared 810 and a fault signal output in dependence on the result of the comparison 812.

There is provided a control system for controlling the configuration of a joint in a surgical robot. The joint is driven by a drivetrain which transfers power from a drive source to the joint. The control system comprises: a joint controller coupled to the joint; and a robot controller (such as a robot arm controller) coupled to the surgical robot. The control system is configured to: receive a drive source configuration signal indicating a configuration of the drive source; receive a joint configuration signal indicating a configuration of the joint; determine a configuration error from a difference in configurations indicated by the drive source configuration signal and the joint configuration signal; output a first fault signal where the determined configuration error equals or exceeds a first configuration error limit determined from a measured joint torque of the joint and a joint stiffness of the joint, the first configuration error limit being associated with one of the joint controller and the robot arm controller; and output a second fault signal where the determined configuration error equals or exceeds a predetermined second configuration error limit, the second configuration error limit being associated with the other of the joint controller and the robot arm controller.

As discussed herein, this approach enables a quicker recovery of the surgical robot from a less serious fault condition, which can reduce delays in operation of the robot, and can reduce the burden on a surgical team in the operating room.

The predetermined second configuration error limit is associated with the joint controller. The determined first configuration error limit is associated with the robot controller. The predetermined second configuration error limit is greater than the determined first configuration error limit. ‘Greater’ here can mean ‘more positive’ or ‘more negative’. I.e. the determined first configuration error limit is within the predetermined second configuration error limit. Thus the robot controller is configured to detect fault conditions sooner than the joint controller.

A first response can be caused, for example by the control system, to the first fault signal. A different, second response can be caused, for example by the control system, to the second fault signal. Suitably, the first response has a lesser impact on the functioning of the control system than the second response. For example, the control system can be configured to restore full control of the surgical robot more quickly following the first response than following the second response. The control system can be configured to restore full control of the surgical robot in fewer steps following the first response than following the second response. The first response may comprise one or more of: warning an operator of the surgical robot that the determined first configuration error limit has been reached or exceeded; and disabling operative control of the joint, and requiring a command to re-enable operative control of the joint. The second response may comprise: disabling operative control of the joint, and requiring a setup procedure to re-enable operative control of the joint.

A method of controlling the configuration of a joint in a surgical robot will now be described with reference to FIG. 9. At 902, a drive source configuration signal is received, for example at a control system. The drive source configuration signal is indicative of the configuration of the drive source. For example, the drive source configuration signal can indicate a position of the drive source. The drive source configuration signal can comprise a measure of the position of the drive source. A joint configuration signal is received, at 904. The joint configuration signal is suitably received at the control system. The joint configuration signal is indicative of the configuration of the joint. For example, the joint configuration signal can indicate a position of the joint. The joint configuration signal can comprise a measure of the position of the joint.

A configuration error is determined 906 from the difference between the configurations indicated in the drive source configuration signal and in the joint configuration signal. Suitably, the configuration error is determined from the difference in the position determined from the drive source and the position determined from the joint.

At 908, a first fault signal is output where the determined configuration error equals or exceeds a first configuration error limit. Suitably the first configuration error limit is determined from a measured joint torque of the joint and a joint stiffness of the joint. The first configuration error limit is associated with one of a joint controller and a robot arm controller. The control system can comprise at least one of the joint controller and the robot arm controller.

At 910, a second fault signal is output, where the determined configuration error equals or exceeds a predetermined second configuration error limit. The second configuration error limit is associated with the other of the joint controller and the robot arm controller.

It will be understood that the method described with reference to FIG. 8 and the method described with reference to FIG. 9 can be implemented separately or together. For example, outputting the first fault signal can comprise steps of the method described with reference to FIG. 8. Similarly, in an example, outputting the second fault signal can comprise steps of the method described with reference to FIG. 8. This can increase the usefulness of the control method.

Similarly, the controller discussed herein can form part of the control system discussed herein. For example, the joint controller can comprise the controller. The robot controller can comprise the controller. That is, the joint controller and/or the robot controller can comprise aspects of the controller described herein. Thus, one or both of the joint controller and the robot controller is suitably enabled to compare a determined configuration error with a configuration error limit, and to output a fault signal in response to a result of the comparison. Thus, either of the joint controller or the robot controller can, e.g. independently of one another, cause the fault signal to be output based on a comparison of a measured configuration error with a configuration error limit. The joint controller and/or the robot controller can be configured to determine the configuration error limit. Suitably, both the joint controller and the robot controller are configured to cause respective fault signals to be output based on respective comparisons of a measured configuration error with respective configuration error limits. Thus, the benefits of both systems can be obtained separately and/or together.

FIGS. 2 to 9, as described above, refer to the forces generated by joints as being torques. Thus, FIGS. 2 to 9 refer to the controllers for controlling the configuration of a rotary joint. In an alternative example, the joint to be controlled may be configured to provide linear motion. In this example, corresponding forces can be measured and calculated using strain gauges or other force sensors.

Reference is now made to FIG. 10 which illustrates various components of an exemplary computing-based device 1000 which may be implemented as any form of a computing and/or electronic device, and in which embodiments of the methods and modification systems described herein may be implemented. The computing-based device 1000 comprises one or more processor 1002 which may be microprocessors, controllers or any other suitable type of processors for processing computer executable instructions. In some examples, for example where a system on a chip architecture is used, the processors 1002 may include one or more fixed function blocks (also referred to as accelerators) which implement a part of the method of modifying a data stream in hardware (rather than software or firmware). Platform software comprising an operating system 1004 or any other suitable platform software may be provided at the computing-based device to enable application software, such as software 1005 implementing the method of FIG. 8 and/or FIG. 9, to be executed on the device.

The computer executable instructions may be provided using any computer-readable media that is accessible by computing-based device 1000. Computer-readable media may include, for example, computer storage media such as memory 1006 and communications media. Computer storage media (i.e. non-transitory machine-readable media), such as memory 1006, includes volatile and non-volatile, removable and non-removable media implemented in any method or technology for storage of information such as computer readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing-based device. In contrast, communication media may embody computer readable instructions, data structures, program modules, or other data in a modulated data signal, such as a carrier wave, or other transport mechanism. As defined herein, computer storage media does not include communication media. Although the computer storage media (i.e. non-transitory machine-readable media, e.g. memory 1006) is shown within the computing-based device 1000 it will be appreciated that the storage may be distributed or located remotely and accessed via a network or other communication link (e.g. using communication interface 1008).

The computing-based device 1000 also comprises an input/output controller 1010 arranged to output display information to a display device 1012 which may be separate from or integral to the computing-based device 1000. The display information may provide a graphical user interface. The input/output controller 1010 is also arranged to receive and process input from one or more devices, such as a user input device 1014 (e.g. a mouse or a keyboard). This user input may be used to initiate verification. In an embodiment the display device 1012 may also act as the user input device 1014 if it is a touch sensitive display device. The input/output controller 1010 may also output data to devices other than the display device, e.g. a locally connected printing device (not shown).

In the description above actions taken by the system have been split into functional blocks or modules for ease of explanation. In practice, two or more of these blocks could be architecturally combined. The functions could also be split into different functional blocks.

The present techniques have been described in the context of surgical robotic systems, though at least some features described are not limited to such systems, but may be applied to robotic systems more generally. In some examples, the present techniques may be applied to robotic systems that operate remotely. Some examples of situations in which the present techniques may be useful include those that make use of ‘snake-like’ robots for exploration, investigation or repair. In the case of a surgical robot the end effector could be a surgical tool such as a scissors, surgical cutter, surgical pincer or cauteriser.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A controller for controlling the configuration of a joint in a surgical robot, the joint being driven by a drivetrain which transfers power from a drive source to the joint, the controller being configured to:
receive a drive source configuration signal indicating a configuration of the drive source;
receive a joint configuration signal indicating a configuration of the joint;
determine a configuration error from a difference in configurations indicated by the drive source configuration signal and the joint configuration signal;
determine a configuration error limit from a measured joint torque of the joint and a joint stiffness of the joint;
compare the determined configuration error and the determined configuration error limit; and
output a fault signal where the determined configuration error equals or exceeds the determined configuration error limit.

2. The controller as claimed in claim 1, in which:
the configuration error limit is joint-specific; and/or
the joint stiffness is joint-specific.

3. The controller as claimed in claim 1, in which the joint stiffness of the joint comprises a minimum joint stiffness for that joint, and optionally in which the minimum joint stiffness is predetermined.

4. The controller as claimed in claim 1, in which:
the joint configuration signal is received from an output position sensor, and/or
the drive source configuration signal is received from an input position sensor.

5. The controller as claimed in claim 1, in which:
where the measured joint torque equals or exceeds a threshold joint torque, a positive configuration error limit is based on the measured joint torque of the joint and the joint stiffness of the joint; and
where the measured joint torque is less than the threshold joint torque, the positive configuration error limit has a predetermined value;
and optionally in which the positive configuration error limit is capped at a maximum allowed configuration error limit.

6. The controller as claimed in claim 1, in which:
where the measured joint torque is equal to or less than a further threshold joint torque, a negative configuration error limit is based on the measured joint torque of the joint and the joint stiffness of the joint; and
where the measured joint torque is greater than the further threshold joint torque, the negative configuration error limit has a predetermined value;
and optionally in which the negative configuration error limit is capped at a minimum allowed configuration error limit.

7. The controller as claimed in claim 1, in which:
the measured joint torque of the joint is a raw torque applied at the joint; and/or
the drive source is a motor.

8. The controller as claimed in claim 4, in which:
the output position sensor is located at, or at a position proximal to, a distal end of the drive train at which the joint is located; and/or
the input position sensor is located at, or at a position proximal to, a proximal end of the drive train at which the drive source is located.

9. The controller as claimed in claim 1, configured to compare the measured joint torque of the joint with a torque threshold, and to output an additional signal where the measured joint torque equals or exceeds the torque threshold.

10. The control system comprising a controller as claimed in claim 1 and a further controller, the further controller being configured to compare the determined configuration error with a predetermined further configuration error limit, and output a further fault signal where the determined configuration error equals or exceeds the predetermined further configuration error limit.

11. A method of controlling the configuration of a joint in a surgical robot, the joint being driven by a drivetrain which transfers power from a drive source to the joint, the method comprising:
receiving a drive source configuration signal indicating a configuration of the drive source;
receiving a joint configuration signal indicating a configuration of the joint;
determining a configuration error from a difference in configurations indicated by the drive source configuration signal and the joint configuration signal;
determining a configuration error limit from a measured joint torque of the joint and a joint stiffness of the joint;
comparing the determined configuration error and the determined configuration error limit; and
outputting a fault signal where the determined configuration error equals or exceeds the determined configuration error limit.

12. The method as claimed in claim 11, further comprising outputting a further fault signal where the determined configuration error equals or exceeds a predetermined further configuration error limit.

13. A control system for controlling the configuration of a joint in a surgical robot, the joint being driven by a drivetrain which transfers power from a drive source to the joint, the control system comprising:
a joint controller coupled to the joint; and
a robot controller coupled to the surgical robot;
the control system being configured to:
receive a drive source configuration signal indicating a configuration of the drive source;
receive a joint configuration signal indicating a configuration of the joint;
determine a configuration error from a difference in configurations indicated by the drive source configuration signal and the joint configuration signal;
output a first fault signal where the determined configuration error equals or exceeds a first configuration error limit determined from a measured joint torque of the joint and a joint stiffness of the joint, the first configuration error limit being associated with one of the joint controller and the robot arm controller; and
output a second fault signal where the determined configuration error equals or exceeds a predetermined second configuration error limit, the second configuration error limit being associated with the other of the joint controller and the robot arm controller.

14. The control system as claimed in claim 13, in which:
the predetermined second configuration error limit is associated with the joint controller; and/or
the determined first configuration error limit is associated with the robot controller.

15. The control system as claimed in claim 13, in which the predetermined second configuration error limit is greater than the determined first configuration error limit.

16. The control system as claimed in claim 13, configured to cause a first response to the first fault signal and to cause a different second response to the second fault signal;
and optionally in which the first response has a lesser impact on a functioning of the control system than the second response.

17. The control system as claimed in claim 16, in which:
the control system can restore full control of the surgical robot more quickly following the first response than following the second response; and/or
the control system can restore full control of the surgical robot in fewer steps following the first response than following the second response.

18. The control system as claimed in claim 16, in which the first response comprises one or more of:
warning an operator of the surgical robot that the determined first configuration error limit has been reached or exceeded;
disabling operative control of the joint, and requiring a command to re-enable operative control of the joint.

19. The control system as claimed in claim 16, in which the second response comprises:
disabling operative control of the joint, and requiring a setup procedure to re-enable operative control of the joint.

20. (canceled)
